(19) United States
(12) Patent Application Publication
(10) Pub. No.: US 2025/0230121 A1
(43) Pub. Date: Jul. 17, 2025

(54) TEMPERATURE CONTROL IN THE PREPARATION OF ALICYCLIC POLYCARBOXYLIC ACIDS AND ESTERS THEREOF

(71) Applicant: Evonik Oxeno GmbH & Co. KG, Marl (DE)

(72) Inventors: Grzegorz Ziomek, Recklinghausen (DE); Thomas Schneider, Schermbeck (DE); Michael Grass, Haltern am See (DE); Berno Simon, Marl (DE)

(73) Assignee: Evonik Oxeno GmbH & Co. KG, Marl (DE)

(21) Appl. No.: 19/021,357

(22) Filed: Jan. 15, 2025

(30) Foreign Application Priority Data
Jan. 16, 2024 (EP) 24152111.1

(51) Int. Cl. C07C 67/303

(52) U.S. Cl. CPC C07C 67/303

(57) ABSTRACT

In the technical field of the preparation of alicyclic compounds, a ring hydrogenation of aromatic compounds prepares alicyclic compounds in a process for preparing alicyclic compounds. The alicyclic compounds may preferably be alicyclic carboxylic acids and esters thereof. An apparatus may be provided for carrying out this process.

### CROSS-REFERENCE TO RELATED APPLICATIONS

This patent application claims priority to European Patent Application No. 24152111.1, filed on Jan. 16, 2024, in the European Patent Office, the entire disclosure of which is hereby incorporated by reference herein.

### BACKGROUND OF THE INVENTION

### Field of the Invention

The invention is in the technical field of the preparation of alicyclic compounds by ring hydrogenation of aromatic compounds. The invention provides processes for preparing alicyclic compounds, preferably alicyclic carboxylic acids and esters thereof, and apparatuses for carrying out this process.

### Description of Related Art

Alicyclic polycarboxylic esters, such as for example the esters of cyclohexane-1,2-dicarboxylic acid, are used as lubricating oil component and as aids in metal processing. They are also used as plasticizers for polyolefins and for PVC.

For the plasticizing of PVC, esters of phthalic acid, such as for example dibutyl, dioctyl, dinonyl or didecyl esters, are predominantly used. The use of these phthalates is becoming increasingly controversial with the public and the use in plastics might be restricted. Alicyclic polycarboxylic esters, of which some have already been described in the literature as plasticizers for plastics, can be a suitable choice for possible substitutes for the plasticizers that are subject to restrictions.

In most cases, the most economical route for the preparation of alicyclic polycarboxylic esters is the ring hydrogenation of the corresponding aromatic polycarboxylic esters, for example the above-mentioned phthalates. For this, a number of processes are already known to those skilled in the art.

For example, application EP 1 676 829 discloses a process for continuous catalytic hydrogenation in at least two series-connected reactors, wherein the catalyst volume is kept as low as possible. The first reactor is operated in loop mode and at least one further reactor is operated in straight pass mode. Over the course of the continuous hydrogenation, the temperature at the outlet of the first reactor, which is operated in loop mode, rises. When the mixture obtained is subsequently supplied at this obtained temperature to the reactor which is operated in straight pass mode, the situation may arise where the temperature in the second reactor becomes too high and the reaction must be shut down.

### SUMMARY OF THE INVENTION

It was therefore an object of the present invention to provide a process for the catalytic hydrogenation of aromatic compounds, preferably of aromatic polycarboxylic acids and esters thereof, which has a high yield and in which the temperature in the second reactor can be controlled.

### BRIEF DESCRIPTION OF THE DRAWINGS

FIG. 1 shows an apparatus for preparing one or more alicyclic compounds.

FIG. 2 shows an alternative embodiment which to a large extent corresponds to the embodiment according to FIG. 1.

### DETAILED DESCRIPTION OF THE INVENTION

This object has been achieved by providing a process for preparing one or more alicyclic compounds, comprising the steps of:
i. providing a stream A, comprising one or more aromatic compounds and a hydrogen-containing hydrogenation gas;
ii. supplying the stream A at a temperature T₁ to a first hydrogenation unit and hydrogenating the one or more aromatic compounds to give one or more corresponding alicyclic compounds;
iii. obtaining a mixture as first product stream at a temperature T₂ comprising aromatic compounds and alicyclic compounds;
iv. separating the product stream obtained in step iii. into a substream which is supplied to stream A in step i. and is hydrogenated again in step ii. as part of stream A, and a substream which is supplied as stream B at a temperature T₃ to one or more further hydrogenation units;
v. hydrogenating the aromatic compounds present in stream B to give the corresponding alicyclic compounds in the one or more further hydrogenation units, and
vi. obtaining a second product stream at a temperature T₄ comprising one or more alicyclic compounds corresponding to the one or more aromatic compounds provided in step i.,
   wherein the temperature T₂ is not the same as the temperature T₃, and preferably the temperature T₂ is higher than T₃.

In the context of the present invention, the term “alicyclic compounds” are to be understood as meaning those compounds which have a saturated ring system having an aliphatic structure. Such compounds are also known as cycloaliphatic compounds. Preferably, the alicyclic compounds that are obtained as products in the context of the present invention have a cyclohexane ring.

In the context of the present invention, “aromatic compounds” are to be understood as meaning those compounds which have at least one ring system which in accordance with Hückel's rule contains a number of 4n+2 delocalized electrons in conjugated double bonds, free electron pairs or unoccupied p-orbitals. Preferably, the aromatic compounds that are used as starting materials in the context of the present invention have a benzene ring.

A “hydrogen-containing hydrogenation gas” is a gas containing hydrogen. In the context of the reaction underlying the present invention, hydrogen is used as a further starting material in addition to the aromatic compounds. In the hydrogenation reaction carried out, the double bonds in the ring of the aromatic compounds used, preferably of the benzene ring, are hydrogenated by an addition reaction of the hydrogen and thereby broken. The reaction takes place in the presence of a solid catalyst. The hydrogen molecule in the hydrogen-containing hydrogenation gas intermediately bonds to the metal atom of the catalyst and the bond between the two hydrogen atoms in the hydrogen molecule is weakened and can interact with an electron-rich multiple bond (double bond). The hydrogenation takes place when two hydrogen atoms are formally transferred to a double bond. This breaks the double bonds in the aromatic compounds and alicyclic compounds are obtained.

The hydrogenation gases used can be any hydrogen-containing gas mixtures which do not contain any harmful amounts of catalyst poisons such as carbon monoxide or hydrogen sulfide. The use of inert gases is optional, and preference is given to using hydrogen in a purity of greater than 95%, in particular greater than 98%. Inert gas components may for example be nitrogen or methane. It is preferable for such an amount of hydrogen to be present in the hydrogenation units that this hydrogen is present in excess, in particular in an excess of 1% to 200%, preferably in an excess of 3% to 100% and particularly preferably in an excess of 5% to 50%, based on the stoichiometric amount needed to achieve the desired conversion or the conversion that is possible in the hydrogenation unit. Setting a sufficient excess of hydrogen can have advantageous effects on the complete hydrogenation of the aromatic bonds.

The individual hydrogenation units can be charged with fresh hydrogen. However, in order to minimize the hydrogen consumption and the effluent losses resulting from the offgas, it is expedient to use the offgas of one hydrogenation unit as hydrogenation gas for another or the same hydrogenation unit. In addition, the offgas of one hydrogenation unit can be reused as fresh hydrogen after work up. For example, in a process that is carried out in two series-connected hydrogenation units, it is advantageous to feed fresh hydrogen into the first hydrogenation unit and to conduct the offgas of the first hydrogenation unit into the second hydrogenation unit. In this case, starting material and hydrogenation gas flow through the hydrogenation units in opposite sequence. It is advantageous in this process regime to keep the hydrogen excess, based on the stoichiometrically required amount, below 30%, in particular below 20%.

In the context of the present invention, a “hydrogenation unit” is to be understood as meaning a hydrogenation reactor or two or more series-connected reactors or two or more parallel-connected reactors or a reactor group composed of parallel- and series-connected reactors. It is therefore to be understood as meaning a reactor or a reactor arrangement that can perform the function of a reactor in the process according to the invention.

In the context of the present invention, the term “recycling” or “loop mode” is understood to mean the at least partial returning of the product stream of a hydrogenation unit as part of the input stream to the same hydrogenation unit. This involves splitting the product stream or the mixture obtained from a hydrogenation unit. This means that a substream of the hydrogenation output or product stream of the first hydrogenation unit is conducted into the first hydrogenation unit together with fresh starting material as stream A. The other substream of the hydrogenation output or product stream of the first hydrogenation unit is hydrogenated in a second hydrogenation unit which is preferably operated in straight pass mode. Instead of one large hydrogenation unit in loop mode, it is also possible to use two or more smaller units that are arranged in series or in parallel. Instead of one large hydrogenation unit with straight pass flow, it is likewise possible to operate two or more series- or parallel-connected units. However, preference is given to using just one hydrogenation unit operated in loop mode and one unit operated in straight pass mode. The process can also be carried out in tube bundle reactors.

It is preferred in the context of the process according to the invention when step iv. is carried out with a circulation ratio of 1:10 to 1:50, preferably of 1:10 to 1:40, particularly preferably of 1:30. This ratio means, with the value 1:10, for example, that ten tonnes of the first product stream are supplied back to the top of the first hydrogenation unit and one tonne is supplied to the at least one further hydrogenation unit.

The circulation ratio is preferably set such that an overall conversion of 80% to 99%, preferably of 85% to 97%, is achieved in the first of the series-connected hydrogenation units and a conversion of 80% to 100%, preferably of 85% to 100% is achieved in the second hydrogenation unit, based on the starting concentration of the compound to be hydrogenated at the inlet of the respective hydrogenation unit. If three or more hydrogenation units are used, the conversions need to be adjusted accordingly.

The at least one further hydrogenation unit can also be operated in loop mode, or in straight pass mode, which means that no recycling of the recyclate to the same hydrogenation unit takes place. The at least one further hydrogenation unit is preferably operated in straight pass mode.

The hydrogenation can be conducted in the absence or preferably in the presence of a solvent. Solvents used may be any liquids that form a homogeneous solution with the starting material and product, are inert under hydrogenation conditions and can be easily separated off from the product. The solvent may also be a mixture of two or more substances and may optionally contain water.

For example, it is possible to use the following substances as solvent: straight-chain or cyclic ethers such as tetrahydrofuran or dioxane, and also aliphatic alcohols in which the alkyl radical has 1 to 13 carbon atoms.

Alcohols usable with preference are isopropanol, n-butanol, isobutanol, n-pentanol, 2-ethylhexanol, nonanols, industrial nonanol mixtures, decanol, industrial decanol mixtures, tridecanols.

When alcohols are used as solvent, it may be expedient to use that alcohol or that alcohol mixture that would form in the hydrolysis of the product. This would rule out by-product formation through transesterification. A further preferred solvent is the hydrogenation product itself.

The use of a solvent allows the concentration of aromatic compounds in the reactor feed to be limited, as a result of which better temperature control in the reactor can be achieved. This can minimize side reactions and accordingly bring about an increase in product yield. Preferably, the concentration of aromatic compounds in the reactor feed is between 1% and 35% by weight, in particular between 5% and 25% by weight, based on the total amount of starting material. The desired concentration range in the case of reactors that are operated in loop mode can be adjusted via the circulation ratio (quantitative ratio of recycled hydrogenation output to starting material).

In step i., as starting materials, at least one aromatic compound and also a hydrogen-containing hydrogenation gas are provided and fed into a first hydrogenation unit via the input stream as stream A. Here, fresh starting material is supplied in the input stream and fed to the first hydrogenation unit via the input stream. The input stream has the temperature T₁. The hydrogenation in step ii. then takes place in this first hydrogenation unit and in step iii. at the end of the hydrogenation unit a mixture is obtained as a first product stream containing hydrogenated compounds (alicyclic compounds) and non-hydrogenated compounds (aromatic compounds). In a preferred embodiment, the product stream from the first hydrogenation unit is transported to the subsequent process step by means of a pump. Commercially available pumps can be used here. Suitable pumps are known to those skilled in the art.

This mixture or the first product stream has the temperature T₂ and is then split in step iv. into two substreams, with one substream being fed as stream to stream A and being subjected again to hydrogenation according to step ii together with fresh starting material in the first hydrogenation unit. The second substream is introduced as stream B into a second hydrogenation unit and has the temperature T₃. This is hydrogenated in at least one further hydrogenation unit in step v. so that the starting material which has not been converted in the first hydrogenation unit is hydrogenated in this second hydrogenation unit to give the corresponding alicyclic compounds. The product mixture obtained as stream in step vi. has the temperature T₄.

The separation in step iv. of the process according to the invention is preferably carried out on a known T-piece of a pipeline. Here, valves are advantageously arranged at both ends of the T-piece in order to control the mass flow and thus the separation.

It has been found in the context of the present invention that when the temperatures of the outlet from the first hydrogenation unit T₂ and the inlet temperature T₃ to the at least one further hydrogenation unit are set independently of one another, then the yield over time of the process can be increased and the temperature in the at least one further hydrogenation unit can be controlled.

The second product stream at the outlet of the at least one further hydrogenation unit preferably contains less than 0.3% by mass, preferably less than 0.1% by mass, in particular less than 0.05% by mass, and particularly preferably 0.005% by mass of the aromatic compounds used as starting material.

The process parameters, such as product, by-product and starting material concentration and temperature, are preferably determined by online analysis. Online analysis acquires the respective parameters in real time, preferably in the product output streams of the first (3) and/or each further (12) hydrogenation unit. Preference is given to using a measurement method selected from the group consisting of reaction calorimetry, ATR-FTIR spectroscopy, Raman spectroscopy, IR spectroscopy, UV and/or UV-VIS spectroscopy, or a combination thereof. This setting can also be effected in automated fashion, i.e. with the aid of computer technology.

It is preferred in the context of the present invention that the hydrogenation of the aromatic compounds provided in step i. is effected with the hydrogen-containing gas provided in step i. over one or more solid catalysts arranged in a fixed bed of the hydrogenation units.

It is further preferred that the solid catalyst includes at least one metal from the eighth transition group of the periodic table of the elements. Preference is given to using, as active metals, platinum, rhodium, palladium, cobalt, nickel or ruthenium or a mixture of two or more thereof, with ruthenium being used in particular as active metal.

In addition to the metals already mentioned, at least one metal from the first and/or seventh transition group of the periodic table of the elements is preferably additionally present in the catalysts. Preference is given to using rhenium and/or copper in addition to the metal from the eighth transition group of the periodic table of the elements.

The catalysts used in the context of this process are preferably metals, as defined above, applied to a support material. The support materials used are preferably materials containing micropores (pore diameter less than 2 nm), mesopores (pore diameter of 2 to 50 nm) and macropores (pore diameter greater than 50 nm). For instance, with respect to the pore type, support materials having the following pore combinations are usable:
a) mesopores only,
b) micropores and mesopores,
c) mesopores and macropores,
d) micropores and mesopores and macropores,
e) micropores and macropores.

Preference is given to using, as support materials, activated carbon, silicon carbide, aluminium oxide, silicon oxide, aluminosilicate, titanium dioxide, zirconium dioxide, magnesium oxide and/or zinc oxide, or mixtures thereof.

Preferably used as support materials are solids that are largely inert under hydrogenation conditions. Examples of these are activated carbon, silicon carbide, silicon dioxide, titanium dioxide and/or zirconium dioxide, and mixtures of these compounds. Very particular preference is given to using titanium dioxides as support materials. Titanium dioxide occurs in three polymorphs (anatase, rutile and brookite), of which anatase and rutile are the most common. A preferred support material is Aerolyst 7711® (Evonik Operations GmbH). This support material consists to an extent of 15% to 20% by mass of rutile and 80% to 85% by mass of anatase. Further examples of suitable titanium dioxide support materials are those produced on the basis of titanium oxides from a sulfuric acid process. They generally contain >98% anatase.

It is particularly preferred that the solid catalyst used for the hydrogenation in step ii. and/or v. is a catalyst comprising ruthenium as sole metal and titanium dioxide as support material. In a preferred embodiment, the same catalyst is used for the hydrogenation in step ii. and in step v.; particularly preferably, this is a catalyst comprising ruthenium as sole metal and titanium dioxide as support material.

In the process according to the invention, the hydrogenation in step ii. and/or v. is carried out in the liquid phase or in the gas phase. The hydrogenation can be conducted continuously or batchwise over suspended catalysts or those arranged in piece form in a fixed bed. In the process according to the invention, preference is given to continuous hydrogenation over a catalyst in fixed bed form, in which the product/starting material phase is mainly in the liquid state under the reaction conditions.

It is preferred that the hydrogenation in step ii. and/or v. is carried out at a pressure of 3 to 300 bar, preferably 15 to 200 bar, particularly preferably 50 to 150 bar.

It is further preferred that the hydrogenation in step ii. and/or v. is carried out at a temperature of 50° C. to 250° C., preferably 70° C. to 200° C. This temperature is present after hydrogenation has been effected in the output streams (T₂ and T₄) of the hydrogenation units. Due to the exothermicity of the hydrogenation reaction, the reaction does not take place at a fixed temperature, but rather within a temperature range as described herein. The temperature of the reaction mixture thus rises as it flows through the hydrogenation unit.

According to the present invention, the temperature T₂ is not the same as the temperature T₃, and preferably T₂ is higher than T₃. In the context of the present invention, it has been found that it is particularly advantageous when the output stream having the temperature T₂ is not supplied directly to the at least one further hydrogenation unit but instead is cooled and supplied to the at least one further hydrogenation unit with a lower temperature T₃. It is therefore particularly preferred that the temperature T₂ of the obtained mixture is higher than the temperature T₃ of the mixture which is supplied to the hydrogenation unit in step iv.

The temperature control (T₂≠T₃, preferably T₂>T₃) can be achieved by disposing, between the output stream of the first hydrogenation unit, i.e. the first product stream, and the input stream of the second hydrogenation unit, a cooling apparatus which cools at least part of the output stream in order to achieve in the input stream to the at least one further hydrogenation unit the temperature T₃ that differs from the temperature T₂. Preferably, the cooling is achieved via a heat exchanger. Appropriate apparatuses are familiar to those skilled in the art. Preference is given to the use of a heat exchanger in order to utilize the extracted heat energy elsewhere in the process or in an integrated system of two or more plants.

It is accordingly possible that the output stream or the first product stream from the hydrogenation in step ii. is cooled after emerging from the first hydrogenation unit, i.e. that the first product stream is cooled prior to being separated in step iv. However, it is also possible that only the part of the output stream that is sent to the at least one further hydrogenation unit/the second hydrogenation unit (stream B) is cooled, i.e. the cooling is effected here after the separation in step iv. In the context of the present invention, it is preferable when the first product stream is cooled prior to being separated in step iv. In this case it is particularly preferred if a bypass is provided in the process, with which part of the first product stream from the first hydrogenation unit can be admixed, prior to being cooled, with stream B via a sidestream. Part of the uncooled first product stream can thus be admixed with stream B in to increase the temperature T₃. As a result, the temperature T₃ can be adjusted independently of the cooling and independently of the temperature of stream A.

It is preferred in the context of the process according to the invention that in step i. one or more aromatic carboxylic esters, preferably one or more aromatic mono-, di- and polycarboxylic esters, are provided.

In the context of the process according to the invention, aromatic compounds such as aromatic poly- and/or monocarboxylic acids or derivatives thereof, in particular the alkyl esters thereof, can be converted to give the corresponding alicyclic polycarboxylic acid compounds. Both full esters and partial esters can be hydrogenated. A full ester is understood to be a compound in which all acid groups have been esterified. Partial esters are compounds having at least one free acid group (or possibly anhydride group) and at least one ester group.

If polycarboxylic esters are used in the process according to the invention, these preferably contain 2, 3 or 4 ester functions.

It is preferred in the context of the process according to the invention that in step i. one or more benzene-, diphenyl-, naphthalene-, diphenyl oxide-, anthracenedi- or -polycarboxylic esters are provided. The alicyclic polycarboxylic acids obtained with the process according to the invention or the derivatives thereof consist of one or more C6 rings where appropriate linked by a carbon-carbon bond or fused.

It is further preferred that in step i. one or more aromatic carboxylic esters having an alcohol component selected from the group consisting of branched or unbranched alkoxyalkyl, cycloalkyl and/or alkyl groups having 1 to 25 carbon atoms, preferably C8-C10 phthalate, C8-C10 terephthalate, C8-C10 isophthalate and C8-C10 trimellitate, particularly preferably di-2-ethylhexyl phthalate, diisononyl phthalate, di-2-ethylhexyl terephthalate, diisononyl terephthalate, di-2-ethylhexyl isophthalate, diisononyl isophthalate, tri-2-ethylhexyl trimellitate and triisononyl trimellitate, are provided.

Here, C8 preferably means 2-ethylhexyl or n-octyl, C9 means isononyl and C10 means isodecyl or 2-propylheptyl.

The process is preferably a process for hydrogenating benzene-1,2-, -1,3- or -1,4-dicarboxylic esters, and/or of benzene-1,2,3-, -1,2,4- or -1,3,5-tricarboxylic esters, that is to say the isomers of cyclohexane-1,2-, -1,3- or -1,4-dicarboxylic esters or of cyclohexane-1,2,3-, -1,3,5- or -1,2,4-tricarboxylic esters are obtained.

In the process according to the invention, the esters of the following aromatic carboxylic acids can for example be used: naphthalene-1,2-dicarboxylic acid, naphthalene-1,3-dicarboxylic acid, naphthalene-1,4-dicarboxylic acid, naphthalene-1,5-dicarboxylic acid, naphthalene-1,6-dicarboxylic acid, naphthalene-1,7-dicarboxylic acid, naphthalene-1,8-dicarboxylic acid, phthalic acid (benzene-1,2-dicarboxylic acid), isophthalic acid (benzene-1,3-dicarboxylic acid), terephthalic acid (benzene-1,4-dicarboxylic acid), benzene-1,2,3-tricarboxylic acid, benzene-1,2,4-tricarboxylic acid (trimellitic acid), benzene-1,3,5-tricarboxylic acid (trimesic acid), benzene-1,2,3,4-tetracarboxylic acid. It is also possible to use acids that are formed from the mentioned acids by substitution of one or more hydrogen atoms bonded to the aromatic ring with alkyl, cycloalkyl or alkoxyalkyl groups.

Preference is given to using alkyl, cycloalkyl and alkoxyalkyl esters for example of the abovementioned acids, where these radicals independently comprise 1 to 25, in particular 3 to 15, very particularly 8 to 13 carbon atoms, and in particular 9 carbon atoms. These radicals may be linear or branched. If a starting material has more than one ester group, these radicals may then be identical or different.

Examples of esters of an aromatic polycarboxylic acid that can be used in the process according to the invention include the following compounds: monomethyl terephthalate, dimethyl terephthalate, diethyl terephthalate, di-n-propyl terephthalate, dibutyl terephthalate, diisobutyl terephthalate, di-tert-butyl terephthalate, dipentyl terephthalate, monoglycol terephthalate, diglycol terephthalate, n-octyl terephthalate, diisooctyl terephthalate, di-2-ethylhexyl terephthalate, di-n-nonyl terephthalate, diisononyl terephthalate, di-2-propylheptyl terephthalate, di-n-decyl terephthalate, di-n-undecyl terephthalate, diisodecyl terephthalate, diisododecyl terephthalate, ditridecyl terephthalate, di-n-octadecyl terephthalate, diisooctadecyl terephthalate, di-n-eicosyl terephthalate, monocyclohexyl terephthalate; monomethyl phthalate, dimethyl phthalate, di-n-propyl phthalate, di-n-butyl phthalate, diisobutyl phthalate, di-tert-butyl phthalate, monoglycol phthalate, diglycol phthalate, di-n-octyl phthalate, diisooctyl phthalate, di-2-ethylhexyl phthalate, di-n-nonyl phthalate, diisononyl phthalate, di-n-decyl phthalate, di-2-propylheptyl phthalate, diisodecyl phthalate, di-n-undecyl phthalate, diisoundecyl phthalate, ditridecyl phthalate, di-n-octadecyl phthalate, diisooctadecyl phthalate, di-n-eicosyl phthalate, monocyclohexyl phthalate; dicyclohexyl phthalate, monomethyl isophthalate, dimethyl isophthalate, diethyl isophthalate, di-n-propyl isophthalate, di-n-butyl isophthalate, diisobutyl isophthalate, di-tert-butyl isophthalate, monoglycol isophthalate, diglycol isophthalate, di-n-octyl isophthalate, diisooctyl isophthalate, di-2-ethylhexyl isophthalate, di-n-nonyl isophthalate, diisononyl isophthalate, di-n-decyl isophthalate, diisodecyl isophthalate, di-n-undecyl isophthalate, diisododecyl isophthalate, di-n-dodecyl isophthalate, ditridecyl isophthalate, di-n-octadecyl isophthalate, diisooctadecyl isophthalate, di-n-eicosyl isophthalate, monocyclohexyl isophthalate.

The process according to the invention is also applicable in principle to benzoic acid and esters thereof. This is understood to mean not just alkyl benzoates but also benzoates of diols, such as for example glycol dibenzoate, diethylene glycol benzoate, triethylene glycol dibenzoate or propylene glycol dibenzoate. The alcohol component of the alkyl benzoates can consist of 1 to 25, preferably 8 to 13, carbon atoms and be in each case linear or branched.

On the industrial scale, aromatic esters, in particular full esters, are often preferably prepared from alcohol mixtures. Examples of appropriate alcohol mixtures include:

C5 alcohol mixtures prepared from linear butenes by hydroformylation followed by hydrogenation; C5 alcohol mixtures prepared from butene mixtures containing linear butenes and isobutene by hydroformylation followed by hydrogenation; C6 alcohol mixtures prepared from a pentene or from a mixture of two or more pentenes by hydroformylation followed by hydrogenation; C7 alcohol mixtures prepared from the trimerization of ethylene or dimerization of propylene or a hexene isomer or another mixture of hexene isomers, by hydroformylation followed by hydrogenation; C8 alcohol mixtures, such as 2-ethylhexanol (2 isomers), prepared by aldol condensation of n-butyraldehyde followed by hydrogenation; C9 alcohol mixtures prepared from C4 olefins by dimerization, hydroformylation and hydrogenation. The preparation of the C9 alcohols may proceed from isobutene or from a mixture of linear butenes or from mixtures of linear butenes and isobutene. The C4 olefins may be dimerized with the aid of various catalysts, for example protic acids, zeolites, organometallic nickel compounds or solid nickel-containing catalysts. The C8 olefin mixtures may be hydroformylated with the aid of rhodium or cobalt catalysts. There is therefore a wide variety of industrial C9 alcohol mixtures; C10 alcohol mixtures prepared from tripropylene by hydroformylation followed by hydrogenation; 2-propylheptanol (2 isomers) prepared by aldol condensation of valeraldehyde followed by hydrogenation; C10 alcohol mixtures prepared from a mixture of at least two C5 aldehydes by aldol condensation followed by hydrogenation; C13 alcohol mixtures prepared from hexaethylene, tetrapropylene or tributene by hydroformylation followed by hydrogenation.

Other alcohol mixtures may be obtained by hydroformylation followed by hydrogenation from olefins or olefin mixtures which arise, for example, in Fischer-Tropsch syntheses, in dehydrogenations of hydrocarbons, in metathesis reactions, in the polygas process, or in other industrial processes. Olefin mixtures with olefins of differing carbon numbers may also be used to prepare alcohol mixtures.

In the process according to the invention, any ester mixture prepared from aromatic polycarboxylic acids and the abovementioned alcohol mixtures may be used. According to the invention, preference is given to using esters prepared from phthalic acid or phthalic anhydride and also terephthalic acid or dimethyl terephthalate and a mixture of isomeric alcohols having 4 to 13 carbon atoms.

Preference is given to a process for preparing one or more alicyclic compounds, comprising the steps of:
i. providing a stream A, comprising one or more aromatic compounds selected from the group consisting of esters of phthalic acid, isophthalic acid, terephthalic acid and/or trimellitic acid, particularly preferably selected from the group consisting of dipentyl terephthalate, di-2-ethylhexyl terephthalate, diisononyl terephthalate, dipentyl phthalate, di-2-ethylhexyl phthalate, diisononyl phthalate, dipentyl isophthalate, di-2-ethylhexyl isophthalate, diisononyl isophthalate, tripentyl trimellitate, tri-2-ethylhexyl trimellitate, triisononyl trimellitate, or mixtures thereof,
and a hydrogen-containing hydrogenation gas;
ii. supplying the stream A at a temperature T₁ to a first hydrogenation unit and hydrogenating the one or more aromatic compounds to give one or more corresponding alicyclic compounds;
iii. obtaining a mixture as first product stream at a temperature T₂ comprising aromatic compounds and alicyclic compounds selected from the group consisting of dialkyl cyclohexane-1,2-dicarboxylates, dialkyl cyclohexane-1,3-dicarboxylates, dialkyl cyclohexane-1,4-dicarboxylates and cyclohexane-1,2,4-tricarboxylates, particularly preferably selected from the group consisting of dipentyl cyclohexane-1,4-dicarboxylate, di-2-ethylhexyl cyclohexane-1,4-dicarboxylate, diisononyl cyclohexane-1,4-dicarboxylate, dipentyl cyclohexane-1,2-dicarboxylate, di-2-ethylhexyl cyclohexane-1,2-dicarboxylate, diisononyl cyclohexane-1,2-dicarboxylate, dipentyl cyclohexane-1,3-dicarboxylate, di-2-ethylhexyl cyclohexane-1,3-dicarboxylate, diisononyl cyclohexane-1,3-dicarboxylate, tripentyl cyclohexane-1,2,4-tricarboxylate, tri-2-ethylhexyl cyclohexane-1,2,4-tricarboxylate, triisononyl cyclohexane-1,2,4-tricarboxylate;
iv. separating the product stream obtained in step iii. into a substream which is supplied to stream A in step i. and is hydrogenated again in step ii. as part of stream A, and a substream which is supplied as stream B at a temperature T₃ to one or more further hydrogenation units;
v. hydrogenating the aromatic compounds present in stream B as defined in step i. to give the corresponding alicyclic compounds in the one or more further hydrogenation units, and
vi. obtaining a second product stream at a temperature T₄ comprising one or more alicyclic compounds selected from the group consisting of dialkyl cyclohexane-1,2-dicarboxylates, dialkyl cyclohexane-1,3-dicarboxylates, dialkyl cyclohexane-1,4-dicarboxylates and 1,2,4-trimellitic esters, particularly preferably selected from the group consisting of dipentyl cyclohexane-1,4-dicarboxylate, di-2-ethylhexyl cyclohexane-1,4-dicarboxylate, diisononyl cyclohexane-1,4-dicarboxylate, dipentyl cyclohexane-1,2-dicarboxylate, di-2-ethylhexyl cyclohexane-1,2-dicarboxylate, diisononyl cyclohexane-1,2-dicarboxylate, dipentyl cyclohexane-1,3-dicarboxylate, di-2-ethylhexyl cyclohexane-1,3-dicarboxylate, diisononyl cyclohexane-1,3-dicarboxylate, tripentyl cyclohexane-1,2,4-tricarboxylate, tri-2-ethylhexyl cyclohexane-1,2,4-tricarboxylate, triisononyl cyclohexane-1,2,4-tricarboxylate, corresponding to the one or more aromatic compounds provided in step i.,
wherein the temperature T₂ is not the same as the temperature T₃, and preferably wherein the temperature T₂ is higher than T₃.

The alicyclic compounds present in the second product stream depend on the starting material used. For example, diisononyl cyclohexane-1,2-dicarboxylate is obtained as product when using diisononyl phthalate as starting material.

Particular preference is given to a process for preparing one or more alicyclic compounds, comprising the steps of:
i. providing a stream A containing diisononyl phthalate (DINP) or di-2-ethylhexyl phthalate (DEHP) and a hydrogen-containing hydrogenation gas;
ii. supplying the stream A at a temperature T₁ to a first hydrogenation unit and hydrogenating the one or more aromatic compounds to give one or more corresponding alicyclic compounds;
iii. obtaining a mixture as first product stream at a temperature T₂ comprising diisononyl cyclohexane-1,2-dicarboxylate (DINCH) or di-2-ethylhexyl cyclohexane-1,2-dicarboxylate (DEHCH);
iv. separating the product stream obtained in step iii. into a substream which is supplied to stream A in step i. and is hydrogenated again in step ii. as part of stream A, and a substream which is supplied as stream B at a temperature T₃ to one or more further hydrogenation units;
v. hydrogenating the aromatic compounds present in stream B to give the corresponding alicyclic compounds in the one or more further hydrogenation units, and
vi. obtaining a second product stream at a temperature T₄ comprising diisononyl cyclohexane-1,2-dicarboxylate (DINCH) or di-2-ethylhexyl cyclohexane-1,2-dicarboxylate (DEHCH);
wherein the temperature T₂ is not the same as the temperature T₃, and preferably wherein the temperature T₂ is higher than T₃.

Particular preference is further given to a process for preparing one or more alicyclic compounds, comprising the steps of:
i. providing a stream A containing diisononyl terephthalate or di-2-ethylhexyl terephthalate and a hydrogen-containing hydrogenation gas;
ii. supplying the stream A at a temperature T₁ to a first hydrogenation unit and hydrogenating the one or more aromatic compounds to give one or more corresponding alicyclic compounds;
iii. obtaining a mixture as first product stream at a temperature T₂ comprising diisononyl cyclohexane-1,4-dicarboxylate or di-2-ethylhexyl cyclohexane-1,4-dicarboxylate;
iv. separating the product stream obtained in step iii. into a substream which is supplied to stream A in step i. and is hydrogenated again in step ii. as part of stream A, and a substream which is supplied as stream B at a temperature T₃ to one or more further hydrogenation units;
v. hydrogenating the aromatic compounds present in stream B to give the corresponding alicyclic compounds in the one or more further hydrogenation units, and
vi. obtaining a second product stream at a temperature T₄ comprising diisononyl cyclohexane-1,4-dicarboxylate or di-2-ethylhexyl cyclohexane-1,4-dicarboxylate;
wherein the temperature T₂ is not the same as the temperature T₃, and preferably wherein the temperature T₂ is higher than T₃.

Particular preference is further given to a process for preparing one or more alicyclic compounds, comprising the steps of:
i. providing a stream A containing triisononyl trimellitate (TINTM) or tri-2-ethylhexyl trimellitate (TOTM) and a hydrogen-containing hydrogenation gas;
ii. supplying the stream A at a temperature T₁ to a first hydrogenation unit and hydrogenating the one or more aromatic compounds to give one or more corresponding alicyclic compounds;
iii. obtaining a mixture as first product stream at a temperature T₂ comprising triisononyl cyclohexane-1,2,4-tricarboxylate or tri-2-ethylhexyl cyclohexane-1,2,4-tricarboxylate;
iv. separating the product stream obtained in step iii. into a substream which is supplied to stream A in step i. and is hydrogenated again in step ii. as part of stream A, and a substream which is supplied as stream B at a temperature T₃ to one or more further hydrogenation units;
v. hydrogenating the aromatic compounds present in stream B to give the corresponding alicyclic compounds in the one or more further hydrogenation units, and
vi. obtaining a second product stream at a temperature T₄ comprising triisononyl cyclohexane-1,2,4-tricarboxylate or tri-2-ethylhexyl cyclohexane-1,2,4-tricarboxylate;
wherein the temperature T₂ is not the same as the temperature T₃, and preferably wherein the temperature T₂ is higher than T₃.

The process according to the invention is preferably conducted under the following conditions.

In the feed of the first hydrogenation unit (loop mode), the concentration of the aromatic compounds as starting material is between 5% and 30% by mass, in particular between 8% and 15% by mass. In the output stream from the first hydrogenation unit, the concentration of the starting material is between 0.3% and 8% by mass, in particular between 1.5% and 4% by mass. The specific liquid hourly space velocity (LHSV, litres of fresh starting material per litre of catalyst per hour) in the first hydrogenation unit (1) is 0.1 to 5 h⁻¹, in particular 0.5 to 3 h⁻¹.

The surface area loading in the first hydrogenation unit is in the range from 25 to 140 m³/m²/h, in particular in the range from 50 to 90 m³/m²/h.

The average hydrogenation temperatures in the first hydrogenation unit are 70 to 150° C., in particular 80 to 120° C.

The hydrogenation pressure in the first hydrogenation unit is 25 to 200 bar, in particular 80 to 110 bar.

The specific liquid hourly space velocity of the second hydrogenation unit (litres of fresh starting material per litre of catalyst per hour) is 1 to 8 h⁻¹, in particular 2 to 5 h⁻¹.

In the second hydrogenation unit, the average temperature is between 7° and 150° C., in particular 80 and 120° C.

The hydrogenation pressure in the second hydrogenation unit is 25 to 200 bar, in particular 80 to 100 bar.

The process variants are suitable in particular for the hydrogenation of phthalic esters, especially for diisononyl phthalates (as isomer mixture “diisononyl phthalate”, e.g. VESTINOL 9 from OXENO GmbH) or di-2-ethylhexyl phthalate.

A further aspect of the present invention is that of providing an apparatus for carrying out a process according to the invention, comprising a first hydrogenation unit and one or more further hydrogenation units and one or more heat exchangers, wherein the heat exchanger is arranged such that the output stream is conducted via an input stream into the heat exchanger and the output stream from the heat exchanger has a lower temperature than the input stream and is then supplied via a pipeline as input stream into a further hydrogenation unit and/or is supplied via the stream in turn into one or more further hydrogenation units, preferably wherein the streams 3 and 9 have a different temperature.

Preferably, at least one further heat exchanger can be present, which for example is arranged in the input stream of the at least one further hydrogenation unit.

It is preferred that, in the context of the apparatus according to the invention, the first and/or one of the further hydrogenation units has one or more fixed-bed catalysts, preferably wherein the solid catalyst includes at least one metal from the eighth transition group of the periodic table of the elements, particularly preferably ruthenium. The preferred support material is titanium dioxide. What has been stated herein for the catalysts used applies correspondingly.

It is also preferred that the pipelines of the first hydrogenation unit (1) are arranged such that part of the first product stream (2) from the first hydrogenation unit (1) can be admixed, before passing through the heat exchanger (3), with stream B via a sidestream. As a result, part of the uncooled first product stream can be mixed with stream B to increase the temperature thereof.

It is further preferred that present in the first and/or in one of the further hydrogenation units is a mixture of aromatic compounds and corresponding alicyclic compounds, preferably a mixture of aromatic carboxylic esters and corresponding alicyclic compounds thereof having an alcohol component selected from the group consisting of branched or unbranched alkoxyalkyl, cycloalkyl and/or alkyl groups having 1 to 25 carbon atoms, preferably from C₈-C₁₀ phthalate, C₈-C₁₀ terephthalate, C₈-C₁₀ isophthalate and C₈-C₁₀ trimellitate, particularly preferably di-2-ethylhexyl phthalate, diisononyl phthalate, di-2-ethylhexyl terephthalate, diisononyl terephthalate, di-2-ethylhexyl isophthalate, diisononyl isophthalate, tri-2-ethylhexyl trimellitate and triisononyl trimellitate, diisononyl phthalate and/or didecyl phthalate and diisononyl cyclohexanedicarboxylates and/or didecyl cyclohexanedicarboxylates, and the corresponding alicyclic compounds thereof. What has been stated herein for the starting materials (aromatic compounds) and products (alicyclic compounds) applies correspondingly.

Preference in the context of the present invention is given to the use of the alicyclic polycarboxylic esters prepared according to the invention as plasticizers in plastics. Preferred plastics are PVC, homo- or copolymers based on ethylene, propylene, butadiene, vinyl acetate, glycidyl acrylate, glycidyl methacrylate, acrylates, acrylates with alkyl radicals, bonded to the oxygen atom of the ester group, of branched or unbranched alcohols having one to ten carbon atom(s), styrene, acrylonitrile or homo- or copolymers of cyclic olefins.

In addition to the abovementioned applications, the alicyclic polycarboxylic esters prepared according to the invention can be used as a lubricating oil component, as a constituent of cooling liquids and metal machining liquids. They can also be used as a component in paints, coatings, inks and adhesives.

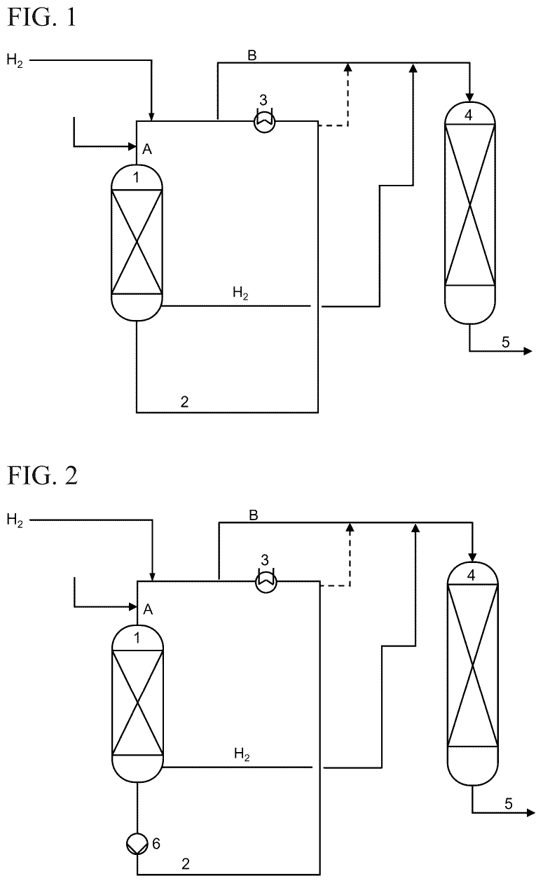

The process according to the invention will for example be described below using the example of the setups shown in FIGS. 1 and 2. The embodiments shown in FIGS. 1 and 2 are exemplary embodiments which are not intended to limit the invention.

FIG. 1 shows an embodiment where in step i. at least one aromatic compound and a hydrogen-containing hydrogenation gas are provided and supplied to a first hydrogenation unit (1) via the input stream as stream A. Stream A has the temperature T₁. The hydrogenation in step ii. is then carried out in this first hydrogenation unit (1) and in step iii. at the end of the hydrogenation unit (1) a mixture comprising hydrogenated compounds (alicyclic compounds) and unhydrogenated compounds (aromatic compounds) is obtained as first product stream (2). This mixture/the first product stream (2) has the temperature T₂ and is preferably cooled in a cooling apparatus (3), for example a heat exchanger (3). In step iv. the cooled product stream is subsequently divided into two substreams, wherein the one substream is subjected as fresh starting material stream A in the first hydrogenation unit (1) to an (optionally renewed) hydrogenation according to step ii. The second substream is introduced as stream B into a second hydrogenation unit (4) and has the temperature T₃. This stream B is admixed with hydrogen-containing hydrogenation gas from the first hydrogenation unit (1). Said stream is hydrogenated in at least one further hydrogenation unit (4) in step v. with the result that any starting material unconverted in the first hydrogenation unit (1) is hydrogenated to afford the corresponding alicyclic compounds in this second hydrogenation unit (4). The product mixture obtained as second product stream (5) in step vi. has the temperature T₄. In the embodiment a bypass may be present, with which part of the first product stream (2) from the first hydrogenation unit (1) may be admixed with stream B via a sidestream, as indicated by the dashed arrow. This makes it possible to adjust the temperature T₃ independently of the cooling.

FIG. 2 shows an alternative embodiment which to a large extent corresponds to the embodiment according to FIG. 1. The only difference is that the first product stream (2) is passed to the cooling apparatus/the heat exchanger (3) with a pump (6).

The product stream (5) at the outlet of the second hydrogenation unit (4) preferably contains less than 0.3% by mass, preferably less than 0.1% by mass, especially less than 0.05% by mass, particularly preferably 0.005% by mass, of alicyclic compounds employed as starting material.

The process parameters, such as product, by-product and starting material concentration and temperature, are preferably determined by online analysis. Online analysis captures the respective parameters in real time, preferably in the product output streams of the first (1) and/or each further hydrogenation unit (4). Preference is given to using a method of measurement selected from the group consisting of reaction calorimetry, ATR-FTIR spectroscopy, Raman spectroscopy, IR spectroscopy, UV and/or UV-VIS spectroscopy or combinations thereof. The process parameters determined therewith make it possible after earlier determination of a threshold value to effect targeted adjustment of the temperature T₁. This adjustment may also be effected in automated fashion, i.e. with the aid of computer technology.

It is preferable in the context of the present invention when the hydrogenation of the aromatic compounds provided in step i. is effected with the hydrogen-containing gas provided in step i. over one or more solid catalysts arranged in a fixed bed of the hydrogenation units.

It is further preferable when the solid catalyst comprises at least one metal from the eighth transition group of the periodic table of the elements. It is preferable to employ, as active metals, platinum, rhodium, palladium, cobalt, nickel or ruthenium or a mixture of two or more thereof, wherein especially ruthenium is employed as the active metal.

In addition to the aforementioned metals, the catalysts may additionally contain at least one metal from the first and/or seventh transition group of the periodic table of the elements. Preference is given to using rhenium and/or copper in addition to the metal from the eighth transition group of the periodic table of the elements.

1. A process for preparing one or more alicyclic compounds, comprising: i. providing a stream A, comprising one or more aromatic compounds and a hydrogen-containing hydrogenation gas; ii. supplying the stream A at a temperature T₁ to a first hydrogenation unit and hydrogenating the one or more aromatic compounds to give one or more corresponding alicyclic compounds; iii. obtaining a mixture as a first product stream at a temperature T₂ comprising one or more aromatic compounds and alicyclic compounds; iv. separating the first product stream obtained in iii. into a substream which is supplied to stream A in i. and is hydrogenated again in ii. as part of stream A, and a substream which is supplied as stream B at a temperature T₃ to one or more further hydrogenation units; v. hydrogenating the one or more aromatic compounds in stream B to give corresponding alicyclic compounds in the one or more further hydrogenation units; and vi. obtaining a second product stream at a temperature T₄ comprising one or more alicyclic compounds corresponding to the one or more aromatic compounds provided in i., wherein the temperature T₂ is not the same as the temperature T₃.

2. The process according to claim 1, comprising:
effecting the hydrogenation of the one or more aromatic compounds provided in i. with the hydrogen-containing gas provided in i. over one or more solid catalysts arranged in a fixed bed of the first hydrogenation unit and/or the one or more further hydrogenation units.

3. The process according to claim 2, wherein the one or more solid catalysts include at least one metal from the eighth transition group of the periodic table of the elements.

4. The process according to claim 1, comprising:
carrying out ii. with a circulation ratio of 1:10 to 1:50.

5. The process according to claim 1, comprising:
carrying out the hydrogenation in ii. and/or iv. at a pressure of 3 to 300 bar.

6. The process according to claim 1, comprising:
carrying out the hydrogenation in ii. and/or iv. at a temperature of 50° C. to 250° C.

7. The process according to claim 1, comprising:
cooling the first product stream prior to being separated in iv.

8. The process according to claim 7, comprising:
providing a bypass, with which part of the first product stream from the first hydrogenation unit is configured to be admixed, prior to being cooled, with stream B via a sidestream.

9. The process according to claim 1, comprising:
providing in i. one or more aromatic carboxylic esters.

10. The process according to claim 1, comprising:
providing in i. one or more benzene-, diphenyl-, naphthalene-, diphenyl oxide-, anthracenedi- or-polycarboxylic esters.

11. The process according to claim 1, comprising:
providing in i. one or more aromatic carboxylic esters having an alcohol component selected from the group consisting of branched or unbranched alkoxyalkyl, cycloalkyl, alkyl groups having 1 to 25 carbon atoms, and mixtures thereof.

12. An apparatus for carrying out a process according to claim 1, comprising:
a first hydrogenation unit,
one or more further hydrogenation units, and
one or more heat exchangers,
wherein the one or more heat exchangers are arranged that the first product stream is conducted into the one or more heat exchangers and an output stream from the one or more heat exchangers has a lower temperature than stream A and is then partially supplied via one or more pipelines as stream B into one or more further hydrogenation units.

13. The apparatus according to claim 12, wherein the first and/or one of the one or more further hydrogenation units has one or more fixed-bed catalysts.

14. The apparatus according to claim 12, wherein the one or more pipelines of the first hydrogenation unit are arranged such that part of the first product stream from the first hydrogenation unit is configured to be admixed, before passing through the one or more heat exchangers, with stream B via a secondary stream.

15. The apparatus according to claim 11, wherein present in the first and/or in one of the one or more further hydrogenation units is a mixture of one or more aromatic compounds and corresponding alicyclic compounds.
